(19) United States
(12) Patent Application Publication
(10) Pub. No.: US 2020/0069901 A1
(43) Pub. Date: Mar. 5, 2020

(54) IMPROVEMENTS FOR RESPIRATORY MASKS

(71) Applicant: P & M HEBBARD PTY LTD, Wangaratta (AU)

(72) Inventor: Peter HEBBARD, Wangaratta (AU)

(21) Appl. No.: 16/609,319

(22) PCT Filed: Apr. 9, 2018
(86) PCT No.: PCT/AU2018/050319

§ 371 Date: Oct. 29, 2019

(30) Foreign Application Priority Data
May 8, 2017 (AU) 2017901690
Feb. 14, 2018 (AU) 2018201087

(51) Int. Cl. A61M 16/06; A61M 16/10

(52) U.S. Cl. CPC A61M 16/0611; A61M 16/0672; A61M 16/1005; A61M 16/104

(57) ABSTRACT

A sealing pad (100) for sealing a respiratory mask. The mask is configured to create a sealing interface. The sealing interface encircles the nostrils and includes portions crossing the nasal ridge and the concave portions. The pad has a face-contacting side to sealingly engage the face, and a mask contacting side to sealingly engage the mask. The pad (100) includes a portion for crossing the nasal ridge and portions (113) for crossing concave portions on either side of the nose such that the mask contacting side is engageable with the mask to define the portions of the sealing interface crossing the nasal ridge and the concave portions. The pad carries, or has one or more defined apertures (107) for carrying, one or more tube portions for conveying fluid across the sealing interface.

### FIELD OF THE INVENTION

The invention relates to improvements for respiratory masks.

### BACKGROUND TO THE INVENTION

A respiratory mask is a mask for providing a person with air or other fluid suitable for breathing. Sometimes the fluid is forcefully provided. Various respiratory masks are employed in a wide variety of applications. By way of example, fire fighters sometimes wear such masks to protect themselves from noxious gases and the depletion of oxygen caused by a fire. Others are used to provide pressurised air to relieve the symptoms of sleep apnea. In the surgical environment masks can be used to forcibly control an anesthetised patient's breathing and/or to supply anesthetic.

A typical mask that anesthetists might use is illustrated in FIG. 2. It includes a cuplike portion C for receiving a patient's nose. The free edge of the cuplike portion is egg-shaped and surrounded by a sealing arrangement S in the form of a cushion for sealing against the patient's face. The port P conveys the breathing fluid to and from the patient.

The egg-shaped seal S is configured to form a sealing interface SI (FIG. 1) encircling the patient's nose and mouth. Some masks merely encircle the lower portions of the person's nose.

Forming an effective seal is important. In the context of treating sleep apnea, a leaking seal is annoying. In the context of forcibly controlling an anesthetised patient's breathing it can be positively dangerous. Of course, any leakage is particularly problematic when the mask is being used to monitor a patient's breathing.

Whilst masks of the type illustrated in FIG. 2 have long been common place in surgical settings the present inventor has recognised that the resultant seal is less than perfect. Sometimes one or more tube portions such as those associated with a nasal cannula or a catheter are required to enter the mask. As suggested in FIG. 3, such tube portions T can open voids V between the patient's cheek CH and the seal S thereby breaking the sealing interface SI.

The US patent application published as US 2004/244799 A1 documents one attempt to address this problem. FIGS. 3 to 5 herein are closely based on FIGS. 1 to 3 of that publication. That publication discloses a respective pad P placed on each of the patient's cheeks CH. Each of the pads P includes a respective face contacting side and a respective mask contacting side. Each pad has an elongate body running along the sealing interface SI and transversely bisected by the tube portion T. The thickness of the pad P tapers away from the tube portion T down towards thin ends so as to avoid the formation of the voids V.

The present invention aims to provide improvements in and for the sealing of respiratory masks, or at least to provide an alternative for those concerned with respiratory masks.

### SUMMARY OF THE INVENTION

The present inventor has recognised that the voids V are not the only source of leakage. The present inventor has recognised that about the nasal ridge is another region where leakage sometimes occurs. ‘Nasal ridge’ and variants of the term as used herein take in the uppermost portion of the nose sometimes referred to as the bridge of the nose.

More specifically the present inventor has in fact identified that typically leakage does not occur at the interface of the nasal ridge and the seal S but rather the region of concave curvature at the juncture of the nose and the cheek. Particularly in patients having pronounced facial features, the seal S is unable to deform to adopt a sufficiently convex shape to press firmly into the contours of these concave portions CP of the face. The present inventor has recognised that this is a cause of the leakage. Furthermore, the present inventor has also recognised that the pad P tends to add further concavity to the already concave region on each side of the nose and thus may in fact exacerbate the leakage in those regions.

Accordingly, in view of these insights, the present invention provides a sealing pad for sealing a respiratory mask;

the mask being for use on a human face;

the human face having a nose, nostrils, a nasal ridge, a mouth, a philtrum and on either side of the nose a respective concave portion and a respective cheek;

the mask being configured to create a sealing interface;

the sealing interface encircling the nostrils and including portions crossing the nasal ridge and the concave portions;

the pad having
a face-contacting side to sealingly engage the face; and
a mask contacting side to sealingly engage the mask;

the pad including a portion for crossing the nasal ridge and portions for crossing the concave portions such that the mask contacting side is engageable with the mask to define the portions of the sealing interface crossing the nasal ridge and the concave portions;

the pad carrying, or having one or more defined apertures for carrying, one or more tube portions for conveying fluid across the sealing interface.

Preferably at least one of the tube portion(s), or at least one of the aperture(s), is positioned to overlie one of the cheeks. Most preferably the pad carries, or has defined apertures for carrying, the tube portions to convey, over each of the cheeks, fluid across the sealing interface.

The pad may carry, or have respective defined apertures, for carrying tubes carrying distinct fluids. By way of example one tube might carry oxygen whilst another tube is a gas sampling tube.

The portions for crossing the concave portions may be thicker than the portion for crossing the nasal ridge. The face-contacting side may be at least tacky to seal against the face and remain on the face when the mask is removed. The sealing interface may encircle the nostrils and the mouth. Preferably the pad has two ends. Most preferably each of the ends is arranged to overlie a respective one of the cheeks. The pad may be approximately inverted U-shaped or inverted V-shaped. Preferably the mask is capable of sealing against the face without the pad.

The pad may include a portion shaped to run obliquely downwards from the nasal ridge and turn to run downwardly alongside the philtrum.

Optionally the pad has a free configuration, in which the face-contacting side is at least approximately planar, and being resiliently deformable to an in-use configuration.

Another aspect of the invention provides a method of treatment including utilising the pad;

delivering a first fluid via the tube portion(s); and

delivering a second fluid via the mask.

Another aspect of the invention provides a method of anesthesia including utilising the pad;

delivering oxygen via the tube portion(s); and

delivering anesthetic via the mask.

Another aspect of the invention provides a range of the pads of differing sizes to suit humans of differing sizes.

### BRIEF DESCRIPTION OF THE DRAWINGS

FIG. 1 is a front view of a face;

FIG. 2 is a perspective view of a respiratory mask;

FIG. 3 is a side view of an interface between a mask and a face;

FIG. 4 is a perspective view of a face and a mask;

FIG. 5 is a side view of a face and a mask;

FIG. 6 is a front view of a face configured with a sealing pad and nasal cannula;

FIG. 7 is a front view of a pad in its free configuration; and

FIG. 8 is a front view of a face configured with the sealing pad of FIG. 7 and a nasal cannula.

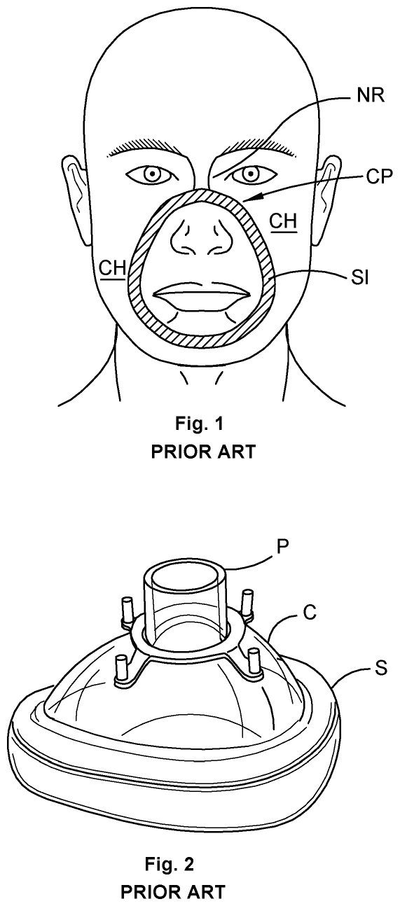

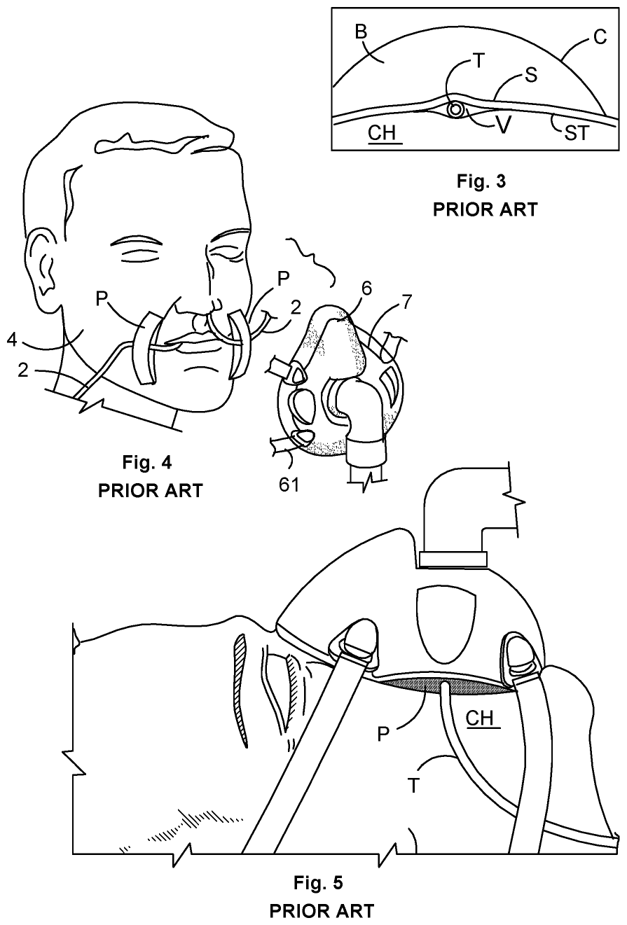

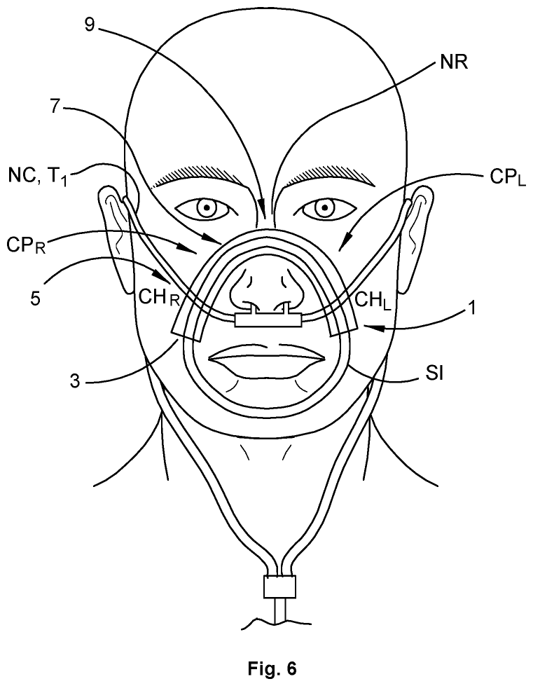

### DETAILED DESCRIPTION

The pad 1 is predominantly formed of a strip of material shaped to upwardly traverse a right cheek CH_{R}, cross a concave portion CP_{R}, cross a nasal ridge NR, cross a concave portion CP_{L} and downwardly traverse a portion of the cheek C_{H}L. As such the pad 1 has the approximate shape of a V, or a U, inverted.

Optionally the pad might encircle the mouth and the nose, although in this variant the pad tapers down to a thin free end 3 on the patient's cheek CH_{R} over which free edge typical seals S are able to seal.

The pad further includes an aperture 5 through which a tubular portion T₁ of the nasal cannula NC passes. The aperture 5 is positioned to cross the sealing interface SI in the vicinity of the patient's cheek CH_{R} so that the tube portion T₁ emerges into the interior of the mask to present the cannula NC for insertion into the nose.

The tube portion T₁ may be permanently bonded within the pad. Alternatively, the pad may be sold separately and the tube portion separately inserted. For this purpose, one of the face contacting side and the mask contacting side of the pad 1 might be penetrated by a slot opening into the aperture 5 to more easily place the tube portion T₁ in to the aperture 5.

Above the aperture 5, the pad 1 includes a concavity-crossing portion 7 leading into a nasal ridge crossing portion 9. The other end of the pad (overlying the other side of the patient's face) is preferably symmetrical to the right-hand end just described.

The portion 7 cooperates with the seal S for improved sealing. The compliance and/or surface properties of the pad 1 may make positive contributions, although it is preferred that the portion 7 is thicker than the portion 9 to go some way to filling in the concave portion CP_{R} to smooth out the curvatures to which the seal S₁ must conform to form the sealing interface SI.

Preferably the face facing side of the pad is at least tacky so that the pad 1, and the tubes carried thereby remain in situ when the mask is removed without requiring the anesthetist's attention. Typically, a small degree of tackiness, as opposed to stronger adhesion, is sufficient for this purpose and is indeed preferred so that post-surgery the pad 1 can be quickly and simply removed without irritation to the patient or requiring any particular effort to clean away residual adhesive.

In surgery, the pad 1 can be used to hold in place an oxygen-delivering nasal cannula NC whilst the patient's breathing is controlled and anesthetic supplied via the mask. It is not uncommon to supply patients with anesthetic via a mask and then once the patient is anesthetised intubate the patient to control their breathing. Supplying the patient with oxygen via a nasal cannula during intubation and extubation can improve health outcomes. The present invention allows for this ongoing supply of oxygen with little or no distraction to the anesthetist or others on the surgical team.

Preferred forms of the pad 1 are formed of soft medical grade silicon, although other materials may be employed. Preferably each of the face contacting side and the mask contacting side of the pad 1 presents a substantially continuous sealing surface, i.e. a surface without any significant breaks along its length through which fluid might leak to or from the mask.

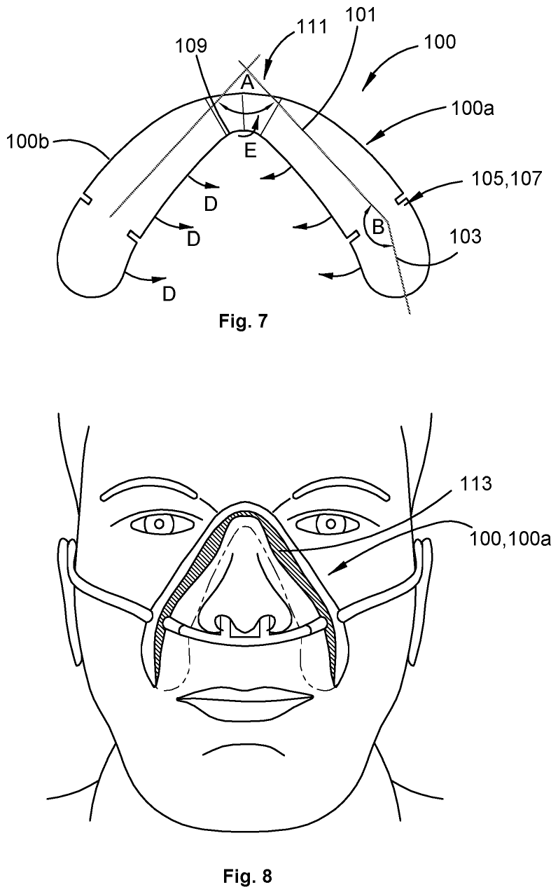

FIGS. 7 and 8 illustrate a preferred pad 100 that substantially consists of a single integral body of material. In this example, the material is silicon and the pad is formed by injection moulding.

FIG. 7 shows the pad 100 in its free configuration. This is the position in which the material is moulded. At its rear, the pad 100 has a face-contacting side that is substantially planar in the free configuration. The pad includes a pair of downwardly divergent arms 100a, 100b. Each of the arms 100a is shaped to run obliquely downwards from the nasal ridge and to turn downwardly to run downwardly alongside the philtrum. In this example, the arm 100a includes an approximately linear upper portion 101 connected to an end portion 103 at a corner defining an included angle B of about 150° so that the arm 100a turns by about 30°. The corner 105 is positioned to sit approximately horizontally adjacent the nostrils. This downward curvature better matches the egg-shaped sealing interface of a cushion mask.

The tube-conveying apertures 107 are coincident with the corners 105.

In its free condition, the portions 101 downwardly diverge from the nasal ridge at an angle A in the vicinity of 90°. To apply the pad 100 to the face, the pad is deformed (as suggested by arrows D) so as to reduce this angle to about 60°. As the arms 100a, 100b move toward each other, an inner edge 109 of the nasal ridge crossing portion 111 deforms away from the face (as suggested by arrow E) whereby the once planar face-contacting side adopts a contoured shape complementary to the bridge of the nose.

The planar face-contacting side lends itself to low-cost manufacture, efficient transport and storage, and to the application of suitable adhesives.

When applied to the face, the once-planar face-contacting side conforms to the contours of the face so that the once-contoured mask-contacting side adopts a shape that (relative to the face per se) has reduced curvature along the line of the sealing interface so as to improve the seal.

FIG. 8 is a cut-away view of the pad showing the thick concave face-contacting portions 113 filling in that curvature so as to reduce leakage in the vicinity of these concaves.

Whilst one or more examples have been described, the invention is not limited to these example(s). Rather, the invention is defined by the claims.

1. A sealing pad for sealing a respiratory mask;
the mask being for use on a human face and capable of sealing against the face without the pad;
the human face having a nose, nostrils, a nasal ridge, a mouth, a philtrum and on either side of the nose a respective concave portion and a respective cheek;
the mask being configured to create a sealing interface;
the sealing interface encircling the nostrils and including portions crossing the nasal ridge and the concave portions;
the pad having
a face-contacting side to sealingly engage the face; and
a mask contacting side to sealingly engage the mask;
the pad including a portion for crossing the nasal ridge and portions for crossing the concave portions such that the mask contacting side is engageable with the mask to define the portions of the sealing interface crossing the nasal ridge and the concave portions;
the pad at least one of
carrying one or more tube portions for conveying fluid across the sealing interface, and
having one or more defined apertures for carrying one or more tube portions for conveying fluid across the sealing interface; and
the portions for crossing the concave portions being thicker than the portion for crossing the nasal ridge.

2. The pad of claim 1 wherein at least one of
at least one of the one or more tube portions is positioned to overlie one of the cheeks, and
at least one of the one or more apertures.

3. The pad of claim 1 at least one of
carrying the tube portions to convey, over each of the cheeks, fluid across the sealing interface, and
having defined apertures for carrying the tube portions to convey, over each of the cheeks, fluid across the sealing interface.

4. The pad of claim 1 wherein the face-contacting side is at least tacky to seal against the face and remain on the face when the mask is removed.

5. The pad of claim 1 wherein the sealing interface encircles the nostrils and the mouth.

6. The pad of claim 1 having two ends each of which is arranged to overlie a respective one of the cheeks.

7. The pad of claim 6 being approximately inverted U-shaped or inverted V-shaped.

8. The pad of claim 1 including a portion shaped to run obliquely downwards from the nasal ridge and turn to run downwardly alongside the philtrum.

9. The pad of claim 1 having a free configuration, in which the face-contacting side is at least approximately planar, and being resiliently deformable to an in-use configuration.

10. A method of treatment comprising
utilising a pad;
the pad being a sealing pad for sealing a respiratory mask;
the mask being for use on a human face and capable of sealing against the face without the pad;
the human face having a nose, nostrils, a nasal ridge, a mouth, a philtrum and on either side of the nose a respective concave portion and a respective cheek;
the mask being configured to create a sealing interface;
the sealing interface encircling the nostrils and including portions crossing the nasal ridge and the concave portions;
the pad having
a face-contacting side to sealingly engage the face; and
a mask contacting side to sealingly engage the mask;
the pad including a portion for crossing the nasal ridge and portions for crossing the concave portions such that the mask contacting side is engageable with the mask to define the portions of the sealing interface crossing the nasal ridge and the concave portions;
the pad at least one of
carrying one or more tube portions for conveying fluid across the sealing interface, and
having one or more defined apertures for carrying one or more tube portions for conveying fluid across the sealing interface; and
the portions for crossing the concave portions being thicker than the portion for crossing the nasal ridge;
the method further comprising
delivering a first fluid via the one or more tube portions; and
delivering a second fluid via the mask.

11. A method of anesthesia comprising
utilising a pad;
the pad being a sealing pad for sealing a respiratory mask;
the mask being for use on a human face and capable of sealing against the face without the pad;
the human face having a nose, nostrils, a nasal ridge, a mouth, a philtrum and on either side of the nose a respective concave portion and a respective cheek;
the mask being configured to create a sealing interface;
the sealing interface encircling the nostrils and including portions crossing the nasal ridge and the concave portions;
the pad having
a face-contacting side to sealingly engage the face; and
a mask contacting side to sealingly engage the mask;
the pad including a portion for crossing the nasal ridge and portions for crossing the concave portions such that the mask contacting side is engageable with the mask to define the portions of the sealing interface crossing the nasal ridge and the concave portions;
the pad at least one of
carrying one or more tube portions for conveying fluid across the sealing interface, and
having one or more defined apertures for carrying one or more tube portions for conveying fluid across the sealing interface; and
the portions for crossing the concave portions being thicker than the portion for crossing the nasal ridge;
the method further comprising
delivering oxygen via the one or more tube portions; and
delivering anesthetic via the mask.

12. A range of pads of differing sizes to suit humans of differing sizes, each of the pads being in accordance with claim 1.
